(19) United States
(12) Patent Application Publication
(10) Pub. No.: US 2009/0182035 A1
(43) Pub. Date: Jul. 16, 2009

(54) USE OF A COMBINATION OF OLOPATADINE AND CILOMILAST TO TREAT NON-INFECTIOUS RHINITIS AND ALLERGIC CONJUNCTIVITIS

(75) Inventors: John M. Yanni, Burleson, TX (US); Daniel A. Gamache, Arlington, TX (US); Steven T. Miller, Arlington, TX (US); Clay Beauregard, Fort Worth, TX (US)

Correspondence Address: ALCON, IP LEGAL, TB4-8, 6201 SOUTH FREEWAY, FORT WORTH, TX, 76134, US

(73) Assignee: Alcon Research, Ltd., Fort Worth, TX (US)

(21) Appl. No.: 12/406,755

(22) Filed: Mar. 18, 2009

Related U.S. Application Data
(63) Continuation-in-part of application No. 12/100,715, filed on Apr. 10, 2008.
(60) Provisional application No. 60/911,176, filed on Apr. 11, 2007.

(51) Int. Cl. A61K 31/335

(52) U.S. Cl. 514/450

(57) ABSTRACT

Disclosed are methods of treating allergic conjunctivitis and non-infectious rhinitis in a subject that involve topically administering to the subject a composition comprising olopatadine and cilomilast.

This application is a continuation in part of U.S. application Ser. No. 12/100,715, filed Apr. 10, 2008.

### BACKGROUND OF THE INVENTION

1. Field of the Invention

The present invention relates generally to the treatment of allergic conjunctivitis and non-infectious rhinitis. More particularly, the present invention concerns methods of treating or preventing allergic conjunctivitis and non-infectious rhinitis in a subject that involve topically administering a composition comprising a pharmaceutically effective amount of olopatadine and cilomilast.

2. Description of Related Art

In industrialized countries, more than 10-15% of the population suffers from allergic rhinitis and/or conjunctivitis. Allergic rhinitis and/or conjunctivitis are type I allergic responses that are mediated by IgE antibodies. As a part of an allergic response to antigen, IgE is generated, which binds to the surface of mast cells and basophils via high affinity Fc receptors that are specific for IgE. Antigen cross-linking the IgE-molecules leads to cellular responses involving release of preformed mediators (e.g., histamine), lipid mediator formation and release, and cytokine generation. Mast cells with their mediators can be regarded as central to the initiation and mediation of allergic inflammation.

Clinical symptoms of allergic rhinitis include sneezing, nasal congestion, nasal itching, and rhinorrhea. Clinical symptoms of allergic conjunctivitis include watery discharge, redness, and edema of the eyelids. These symptoms may vary in intensity from the nuisance level to debilitating.

Allergic rhinitis often coexists with allergic conjunctivitis, and other disorders or conditions, such as asthma, sinusitis, atopic dermatitis, and the presence of nasal polyps. All these can frequently lead to significant impairment of quality of life.

Histamine has been implicated in allergic rhinitis and allergic conjunctivitis. Histamine is an important mediator released from mast cells that populate the walls of the nasal mucous membrane. When released, histamine is known to bind competitively to local histamine H₁ receptors and cause sneezing, nasal itching, and swelling of the nasal membranes. The primary action of antihistamines relates to their ability to bind to H₁ histamine receptors, thereby blocking the ability of histamine to bind to these receptors. Anti-histamine compounds that bind to histamine receptors have been found to be useful in treating the signs and symptoms of these conditions.

Conventional H₁ receptor antagonists (“H₁ antagonists”) are widely used as antihistamine agents for treating allergic conjunctivitis and allergic rhinitis. H₁ antagonists target some of the signs and symptoms including itching, sneezing, and inflammation that are associated with these conditions. One limitation of H₁ receptor antagonists is that they are antihistaminic only, providing primarily short-term relief of symptoms.

Other therapies for allergic rhinitis include leukotriene receptor antagonists, decongestants, nasal corticosteroids, intranasal antihistamines, intranasal cromolyns, and intranasal anticholinergic agents. These therapies have disadvantages, however, including steroid-related side effects (nasal corticosteroids), and absence of a direct anti-histaminic effect (intranasal cromolyns, leukotriene antagonists, and intranasal anticholinergic agents).

Tumor Necrosis Factor α (TNFα) is a cytokine that has been shown to play a pivotal role in immune and inflammatory responses, including allergic rhinitis and conjunctivitis. TNFα is a soluble homotrimer of 17 kD protein subunits (Smith, 1987). TNFα is derived from mononuclear cells and macrophages, along with other cell types. Modulation of TNFα has been proposed as a therapeutic strategy for allergic conjunctivitis, and other conditions associated with activation of TNFα.

The widespread incidence of allergic conjunctivitis and allergic rhinitis means that there is a continuing need for the discovery of therapies that are effective to ameliorate the signs and symptoms of this condition.

### SUMMARY OF THE INVENTION

The present invention overcomes drawbacks of the prior art by providing for methods for treating allergic conjunctivitis and non-infectious rhinitis. In particular, the inventors have found that treatment of non-infectious rhinitis or allergic conjunctivitis with a combination of olopatadine and cilomilast provides both immediate and long-term relief.

The allergic conjunctivitis may be seasonal allergic conjunctivitis, perennial allergic conjunctivitis, vernal conjunctivitis, giant papillary conjunctivitis, or atopic keratoconjunctivitis.

In particular embodiments, the disease to be treated or prevented is allergic conjunctivitis, and administration is topical to the surface of an eye or periocular skin of the eyelids of the subject. In other particular embodiments, the disease to be treated or prevented is allergic rhinitis, and the therapeutic agents are administered topically into the nose, such as by drop or aerosol.

Although a wide variety of treatments for non-infectious rhinitis and allergic conjunctivitis are available, many have significant limitations or side effects. For example, anti-histamine products are anti-histaminic only and do not address the inflammation component of an allergic response, while nasal corticosteroids are associated with steroid side effects. There is a need for more effective therapies for allergic conjunctivitis and non-infectious rhinitis.

Although not wishing to be bound to any theory, it is believed that the combination of olopatadine and cilomilast provides immediate relief from acute allergy effects such as sneezing, edema, nasal itching and rhinorrhea because of olopatadine and protection from allergic inflammation and congestion because of cilomilast. The combination product of the present invention is devoid of the risk of steroid-induced side effects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWING

FIG. 1 shows the acute phase and anti-inflammatory activity of olopatadine and cilomilast in a guinea pig model of passive conjunctival anaphylaxis.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more.

“Treatment” and “treating” refer to administration or application of a therapeutic agent to a subject or performance of a procedure or modality on a subject for the purpose of obtaining a therapeutic benefit of a disease or health-related condition. Treating includes inhibiting the state, disorder or condition, i.e., arresting or reducing the development of the disease or at least one clinical or subclinical symptom thereof, or relieving the disease, i.e., causing regression of the state, disorder or condition or at least one of its clinical or subclinical symptoms. The benefit to a subject to be treated is either statistically significant or at least perceptible to the patient or to the physician. For example, in the context of the present invention, allergic conjunctivitis may be treated by topically applying to the ocular surface a pharmaceutically effective amount of olopatadine and cilomilast to reduce itching, redness, and irritation of the conjunctiva.

The term “therapeutic benefit” or “therapeutically effective” as used throughout this application refers to anything that promotes or enhances the well-being of the subject with respect to the medical treatment of his condition. This includes, but is not limited to, a reduction in the frequency or severity of the signs or symptoms of a disease. For example, regarding the treatment of allergic rhinitis, a therapeutic benefit is obtained when there is decreased rhinorrhea.

A “pharmaceutically effective amount” means the amount of a compound that, when administered to a mammal for treating a state, disorder or condition, is sufficient to effect such treatment. The “pharmaceutically effective amount” will vary depending on the disease and its severity and the age, weight, physical condition and responsiveness of the mammal to be treated.

The methods set forth herein can be applied in the treatment of allergic conjunctivitis or non-infectious rhinitis. Conjunctivitis is an inflammatory disease that affects the conjunctiva of one or both eyes of an individual. Symptoms and signs include redness, tearing, discharge, irritation, and itching of the eyes. The allergic conjunctivitis may be seasonal allergic conjunctivitis, perennial allergic conjunctivitis, giant papillary conjunctivitis, atopic keratoconjunctivitis, or vernal conjunctivitis. Non-infectious rhinitis is inflammation of the lining of the nose, which may be caused by allergies or other factors such as cigarette smoke, changes in temperature, exercise and stress. Rhinitis may also be associated with asthma, sinusitis, atopic dermatitis, and the presence of nasal polyps. Symptoms include sneezing, nasal congestion, nasal itching, and rhinorrhea. The non-infectious rhinitis may be seasonal allergic rhinitis, perennial allergic rhinitis, vasomotor rhinitis, or occupational allergic rhinitis.

According to the present invention, a composition comprising a combination of olopatadine and cilomilast is topically applied. The administration is topical to the eye or nose. As used herein, administration topical to the eye includes topical compositions dropped or placed on the eye or placed underneath the eye lids, as well as compositions applied to the periocular skin and surface of the eyelids. As used herein, administration topical to the nose includes delivering compositions by drop or spray into the nostrils and nasal passages.

Olopatadine is a known anti-allergy compound possessing H₁ antagonist activity. See, for example, U.S. Pat. Nos. 5,116,863 and 5,641,805, the entire contents of which are hereby incorporated by reference.

Cilomilast is a known PDE4 inhibitor. See, for example, U.S. Pat. Nos. 5,552,483 and 6,740,765, the entire contents of which are hereby incorporated by reference.

In general, the concentration of olopatadine in the compositions of the present invention will be from 0.0001% to 1.0% (w/v), preferably from 0.01 to 0.2% (w/v), and most preferably from 0.05 to 0.2% (w/v), while the concentration of cilomilast will be from 0.0001 to 1% (w/v), preferably from 0.001 to 0.2% (w/v), more preferably from 0.01 to 0.1% (w/v), and most preferably 0.05% (w/v). In one preferred embodiment, the concentration of olopatadine is 0.1% (w/v). In another preferred embodiment, the concentration of olopatadine is 0.2% (w/v).

In particular embodiments, the compositions are suitable for topical application to mammalian eyes. For example, for ophthalmic administration, the formulation may be a solution, a suspension, a gel, or an ointment. The compositions are preferably formulated for topical application to the eye in aqueous solution in the form of drops. The term “aqueous” typically denotes an aqueous composition wherein the carrier is to an extent of >50%, more preferably >75% and in particular >90% by weight water. These drops may be delivered from a single dose ampoule which may preferably be sterile and thus rendering bacteriostatic components of the formulation unnecessary. These drops may also be delivered from a multi-dose container, particularly when the composition contains a preservative ingredient. Alternatively, the drops may be delivered from a multi-dose bottle which may preferably comprise a device which extracts preservative from the formulation as it is delivered, such devices being known in the art.

In other aspects, components of the invention may be delivered to the eye as a concentrated gel or similar vehicle which forms dissolvable inserts that are placed beneath the eyelids.

In addition the components can be place onto the outer eye lid and periocular skin in a skin cream, gel, ointment, or lotion formulation.

In addition to the active ingredients, the compositions of the present invention may contain excipients. For example, the compositions may include one or more pharmaceutically acceptable buffering agents, preservatives (including preservative adjuncts), tonicity-adjusting agents, surfactants, solubilizing agents, stabilizing agents, comfort-enhancing agents, polymers, emollients, pH-adjusting agents and/or lubricants.

Suitable buffering agents include phosphates, borates, citrates, acetates and the like. Examples of preservatives include quaternary ammonium compounds, such as benzalkonium chloride, benzododecinium bromide, or polyquaternium-1. Other examples of preservatives include sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, or sorbic acid. Suitable tonicity-adjusting agents include mannitol, sodium chloride, glycerin, sorbitol and the like. Suitable surfactants include ionic and nonionic surfactants, though nonionic surfactants are preferred, such as polysorbates, polyethoxylated castor oil derivatives and oxyethylated tertiary octylphenol formaldehyde polymer (tyloxapol). Suitable chelating agents include sodium edetate and the like. Suitable antioxidants include sulfites, ascorbates, BHA and BHT.

Topical ophthalmic compositions are preferably isotonic, or slightly hypotonic in order to combat any hypertonicity of tears caused by evaporation and/or disease. The compositions of the present invention generally have an osmolality in the range of 220-320 mOsm/kg, and preferably have an osmolality in the range of 235-260 mOsm/kg. The compositions of the invention have a pH in the range of 5-9, preferably 6.5-7.5, and most preferably 6.8-7.4.

In certain embodiments, the therapeutic agents are formulated in a composition that comprises one or more tear substitutes. A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. The formulation of the present invention may be used with contact lenses or other ophthalmic products.

In other embodiments, the compositions of the present invention are administered topically to the nose. Topical nasal compositions are known and include aerosols and aqueous sprays or mists. As in the case of ophthalmic compositions, nasal compositions may contain excipients. For example, the compositions may include one or more pharmaceutically acceptable buffering agents, preservatives (including preservative adjuncts), tonicity-adjusting agents, surfactants, solubilizing agents, stabilizing agents, comfort-enhancing agents, polymers, emollients, pH-adjusting agents and/or lubricants.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Topical Ophthalmic Composition

| Ingredient | Amount (% w/v) |
|---|---|
| Olopatadine | 0.1-0.2 |
| Cilomilast | 0.05 |
| Dibasic Sodium Phosphate or | 0.01-0.5 |
| Tromethamine | |
| Sodium Chloride | 0.1-0.8 |
| Mannitol or Sucrose | 1-5 |
| Polysorbate 80 | 0.01-0.5 |
| NaOH and/or HCl | Adjust pH 7 ± 2 |
| Polyquad* | 0-0.005 |
| Purified Water | Q.S. 100 |

*If composition will be packaged for multi-dose use.

### Example 2

### Topical Nasal Composition

| Ingredient | Amount (% w/v) |
|---|---|
| Olopatadine | 0.6 |
| Cilomilast | 0.1 |
| Dibasic Sodium Phosphate | 0.01-0.5 |
| Sodium Chloride | 0.1-0.8 |
| Mannitol or Sucrose | 1-5 |
| Polysorbate 80 | 0.01-0.5 |
| NaOH and/or HCl | Adjust pH 5.5 ± 2 |
| Benzalkonium Chloride* | 0-0.02 |
| Purified Water | Q.S. 100 |

*If composition will be packaged for multi-dose use

### Example 3

### Ointment Composition

| Ingredient | Amount (% w/v) |
|---|---|
| Olopatadine | 0.1 |
| Cilomilast | 0.05 |
| Mineral Oil | 0.1-0.5 |
| Petrolatum | Q.S. 100 |

### Example 4

### Aqueous Gel Composition

| Ingredient | Amount (% w/v) |
|---|---|
| Olopatadine | 0.2 |
| Cilomilast | 0.05 |
| Carbomer | 0.2-0.5 |
| Benzalkonium Chloride | 0.01 |
| Mannitol | 4.0 |
| NaOH/HCl | Q.S. to pH 5.5-7.5 |
| Purified Water | Q.S. 100 |

### Example 5

### Passive Conjunctival Anaphylaxis

Drugs were prepared in standard ophthalmic suspension vehicle and allowed to vortex overnight. Cilomilast and olopatadine were prepared in the same drop by preparing separate 0.2% formulations and then combining them 50:50 to yield a 0.1% concentration of each drug. In groups where cilomilast and olopatadine were given in consecutive drops, olopatadine was applied first followed 5 minutes later by cilomilast. All topical drops were administered as a single 20 μL application.

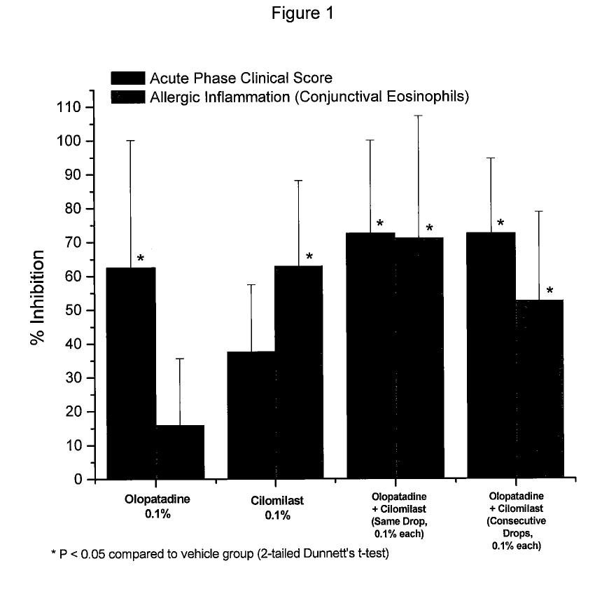

Guinea pigs (male Hartley outbred, 250-300 g) were divided into groups of six. Animals were passively sensitized to ovalbumin by a single subconjunctival injection to the right eye of 10 μL of undiluted anti-ovalbumin guinea pig serum (antiserum). One group was injected with saline only. Twenty-four hours after sensitization all groups were topically challenged with 0.5 mg of ovalbumin in saline to the right eye. Animals were pre-treated with drug or vehicle 60 minutes prior to challenge. Thirty minutes after topical challenge each animal was scored by a masked observer for clinical signs of conjunctivitis, redness/congestion, swelling, and tearing/discharge, with a total score of 0 to 10. Animals were dosed a second time with drug or vehicle 8 hours after challenge. Animals were euthanized 24 hours after challenge and conjunctivae were collected and assayed for eosinophil peroxidase (EPO) activity, an indirect marker of tissue eosinophil concentration. Briefly, tissue samples were weighed frozen, then mechanically homogenized in 50 mM HEPES buffer, pH 6.5, containing 0.5% cetyltrimethylammonium chloride and 6 mM KBr. Homogenates were freeze-thawed three times and sonicated. EPO activity in diluted homogenates was measured by reacting 75 μL of sample supernatant with 75 μL of reaction buffer (50 mM HEPES, pH 6.5, 6 mM KBr, 6 mM o-phenylenediamine, and 8.8 mM H₂O₂ for 3 minutes. The reaction was stopped with equal volume of 4N H₂SO₄ and samples were read on a spectrophotometric plate reader at 490 nm. Concentration of EPO in each sample was calculated from a standard curve generated by reacting recombinant human EPO with the reaction buffer. EPO values for each sample were normalized to tissue weight. Data are expressed as group means±standard deviation. Means are considered significantly different when P<0.05 as determined by Dunnett's two-tailed t-test. The results are shown in Table 1 and in FIG. 1.

TABLE 1
Effects of Olopatadine and Cilomilast on Acute Phase Clinical Score and Allergic
Inflammation in a Guinea Pig Model of Passive Conjunctival Anaphylaxis

| | Clinical Score | | | | EPO Activity (ng EPO/mg tisue) | | | |
|---|---|---|---|---|---|---|---|---|
| Treatment | Mean | ± | SD | % Inhibition | Mean | ± | SD | % Inhibition |
| Unsensitized control | 0 | ± | 0 | | 391.25 | ± | 230.96 | |
| Vehicle only | 6.7 | ± | 1.5 | | 1567.86 | ± | 782.39 | |
| Olopatadine, 0.1% | 2.2 | ± | 1.5 | 67.5* | 1320.23 | ± | 311.59 | 15.8 |
| Cilomilast, 0.1% | 4.2 | ± | 1.3 | 37.5 | 582.26 | ± | 396.68 | 62.8* |
| Olopatadine, 0.1%, | 1.8 | ± | 1.8 | 72.5* | 453.91 | ± | 566.88 | 71.0* |
| and Cilomilast, 0.1% | | | | | | | | |
| (same drop) | | | | | | | | |
| Olopatadine, 0.1%, | 1.8 | ± | 1.5 | 72.5* | 744.71 | ± | 411.52 | 52.5* |
| and Cilomilast, 0.1% | | | | | | | | |
| (consecutive drops) | | | | | | | | |

*P < 0.05 compared to vehicle only group (Dunnett's two-tailed t-test)

Co-administration of olopatadine and cilomilast at 0.1% each provided significant protection of both early and late phase allergic responses. These responses were statistically equivalent with olopatadine alone in early phase activity and cilomilast alone in late phase activity The combinatorial activity of olopatadine and cilomilast was seen with concurrent dosing (same drop) as well as with consecutive dosing 5 min apart.

All of the methods disclosed and claimed herein can be executed without undue experimentation in light of the present disclosure. While the methods of this invention have been described in terms of preferred embodiments, it will be apparent to those of skill in the art that variations may be applied in the steps or in the sequence of steps of the method described herein without departing from the concept, spirit and scope of the invention. More specifically, it will be apparent that certain agents which are both chemically and pharmacologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for treating allergic conjunctivitis in a human subject comprising topically administering to the eye of the subject a composition comprising olopatadine and cilomilast.

2. The method of claim 1, wherein the allergic conjunctivitis is selected from the group consisting of seasonal allergic conjunctivitis; perennial allergic conjunctivitis; giant papillary conjunctivitis; vernal conjunctivitis; and atopic keratoconjunctivitis.

3. The method of claim 1 wherein the composition comprises olopatadine in an amount from 0.0001-1.0% (w/v).

4. The method of claim 3 wherein the composition comprises olopatadine in an amount from 0.01-0.2% (w/v).

5. The method of claim 4 wherein the composition comprises olopatadine in an amount from 0.05-0.2% (w/v).

6. The method of claim 1 wherein the composition comprises cilomilast in an amount from 0.0001-1% (w/v).

7. The method of claim 6 wherein the composition comprises cilomilast in an amount from 0.001-0.2% (w/v).

8. The method of claim 7 wherein the composition comprises cilomilast in an amount from 0.01-0.1% (w/v).

9. A method for treating non-infectious rhinitis in a human subject comprising topically administering to the nose of the subject a composition comprising olopatadine and cilomilast.

10. The method of claim 9, wherein the non-infectious rhinitis is selected from the group consisting of seasonal allergic rhinitis; perennial allergic rhinitis; vasomotor rhinitis; and occupational allergic rhinitis.

11. The method of claim 9 wherein the composition comprises olopatadine in an amount from 0.0001-1.0% (w/v).

12. The method of claim 11 wherein the composition comprises olopatadine in an amount from 0.01-0.2% (w/v).

13. The method of claim 12 wherein the composition comprises olopatadine in an amount from 0.05-0.2% (w/v).

14. The method of claim 9 wherein the composition comprises cilomilast in an amount from 0.0001-1% (w/v).

15. The method of claim 14 wherein the composition comprises cilomilast in an amount from 0.001-0.2% (w/v).

16. The method of claim 15 wherein the composition comprises cilomilast in an amount from 0.01-0.1% (w/v).
